(19) United States
(12) Patent Application Publication
(10) Pub. No.: US 2022/0313480 A1
(43) Pub. Date: Oct. 6, 2022

(54) APPARATUS AND METHOD FOR A HEATING PATCH

(71) Applicant: Alia E. Parker, Chattanooga, TN (US)

(72) Inventor: Alia E. Parker, Chattanooga, TN (US)

(21) Appl. No.: 17/831,970

(22) Filed: Jun. 3, 2022

Related U.S. Application Data
(60) Provisional application No. 63/200,939, filed on Apr. 5, 2021.

(51) Int. Cl. A61F 7/03

(52) U.S. Cl. CPC A61F 7/034; A61F 2007/0226

(57) ABSTRACT

A heating patch assembly adapted for use on a human. The preferred heating patch assembly includes a heating patch assembly lining and a heating patch assembly thermal conductor. The preferred heating patch assembly thermal conductor is disposed within the heating patch assembly lining. The preferred heating patch assembly is adapted to induce vasodilation and increase cardiac output in the human. A method for increasing cardiac output including applying the heating patch assembly to a human chest.

### CROSS-REFERENCES TO RELATED APPLICATIONS/PATENTS

This application relates back to and claims the benefit of priority from U.S. Provisional Application for Patent Ser. No. 63/200,939 titled “Cardiac Output patch” and filed on Apr. 5, 2021.

### FIELD OF THE INVENTION

The present invention relates generally to apparatuses and methods for heating patches, and particularly to apparatuses and methods for heating patches adapted to increase cardiac output.

### BACKGROUND AND DESCRIPTION OF THE PRIOR ART

It is known to use apparatuses and methods to provide heat to a human body from a heating patch. Conventional apparatuses and methods, however, suffer from one or more disadvantages. For example, conventional heating patches and methods are not adapted to increase cardiac output, cardiac index, vasodilation, and coronary artery and overall circulatory system perfusion. Conventional heating patches and methods also do not sufficiently reduce pulmonary hypertension, the force required to open the aortic valve, systemic vascular resistance or afterload, the workload of the heart, or stress. Further, conventional heating patches and methods are undesirably invasive and risky. Still further, conventional heating patches and methods are not designed specifically for post-op heart surgery, cardiogenic shock, or septic shock and do not provide a targeted temperature range or application time. In addition, conventional heating patches and methods do not provide an anti-microbial, copper-infused lining or iron oxidation as a thermal conductor for natural and safe heating.

It would be desirable, therefore, if an apparatus and method for a heating patch could be provided that would be adapted to increase cardiac output, cardiac index, vasodilation, and coronary artery and overall circulatory system perfusion. It would also be desirable if such an apparatus and method for a heating patch could be provided that would sufficiently reduce pulmonary hypertension, the force required to open the aortic valve, systemic vascular resistance or afterload, the workload of the heart, and stress. It would be further desirable if such an apparatus and method for a heating patch could be provided that would not be invasive or undesirably risky. It would be still further desirable if such an apparatus and method for a heating patch could be provided that would be designed specifically for post-op heart surgery, cardiogenic shock, or septic shock and provide a targeted temperature range and application time. In addition, it would be desirable if such an apparatus and method for a heating patch could be provided that would provide an anti-microbial, copper-infused lining and iron oxidation as a thermal conductor for natural and safe heating.

### Advantages of the Preferred Embodiments of the Invention

Accordingly, it is an advantage of the preferred embodiments of the invention claimed herein to provide an apparatus and method for a heating patch that is adapted to increase cardiac output, cardiac index, vasodilation, and coronary artery and overall circulatory system perfusion. It is also an advantage of the preferred embodiments of the invention claimed herein to provide an apparatus and method for a heating patch that sufficiently reduces pulmonary hypertension, the force required to open the aortic valve, systemic vascular resistance or afterload, the workload of the heart, and stress. It is another advantage of the preferred embodiments of the invention claimed herein to provide an apparatus and method for a heating patch that is not invasive or undesirably risky. It is still another advantage of the preferred embodiments of the invention claimed herein to provide an apparatus and method for a heating patch that is designed specifically for post-op heart surgery, cardiogenic shock, or septic shock and provide a targeted temperature range and application time. It is yet another advantage of the preferred embodiments of the invention claimed herein to provide an apparatus and method for a heating patch that provides an anti-microbial, copper-infused lining and iron oxidation as a thermal conductor for natural and safe heating.

Additional advantages of the preferred embodiments of the invention will become apparent from an examination of the drawings and the ensuing description.

### Explanation of the Technical Terms

The use of the terms “a,” “an,” “the,” and similar terms in the context of describing the invention are to be construed to cover both the singular and the plural, unless otherwise indicated herein or clearly contradicted by context. The terms “comprising” “having,” “including,” and “containing” are to be construed as open-ended terms (i.e., meaning “including, but not limited to,”) unless otherwise noted. The terms “substantially,” “generally,” and other words of degree are relative modifiers intended to indicate permissible variation from the characteristic so modified. The use of such terms in describing a physical or functional characteristic of the invention is not intended to limit such characteristic to the absolute value which the term modifies, but rather to provide an approximation of the value of such physical or functional characteristic. All methods described herein can be performed in any suitable order unless otherwise specified herein or clearly indicated by context.

Terms concerning attachments, coupling and the like, such as “attached,” “connected,” and “interconnected,” refer to a relationship wherein structures are secured or attached to one another either directly or indirectly through intervening structures, as well as both moveable and rigid attachments or relationships, unless specified herein or clearly indicated by context. The term “operatively connected” is such an attachment, coupling or connection that allows the pertinent structures to operate as intended by virtue of that relationship.

The use of any and all examples or exemplary language (e.g., “such as,” “preferred,” and “preferably”) herein is intended merely to better illuminate the invention and the preferred embodiments thereof, and not to place a limitation on the scope of the invention. Nothing in the specification should be construed as indicating any element as essential to the practice of the invention unless so stated with specificity.

### SUMMARY OF THE INVENTION

The apparatus of the invention comprises a heating patch assembly adapted for use on a human. The preferred heating patch assembly comprises a heating patch assembly lining and a heating patch assembly thermal conductor. The preferred heating patch assembly thermal conductor is disposed within the heating patch assembly lining. The preferred heating patch assembly is adapted to induce vasodilation and increase cardiac output in the human.

The method of the invention comprises a method for increasing cardiac output. The preferred method comprises providing a heating patch assembly adapted for use on a human. The preferred heating patch assembly comprises a heating patch assembly lining and a heating patch assembly thermal conductor. The preferred heating patch assembly thermal conductor is disposed within the heating patch assembly lining. The preferred heating patch assembly is adapted to induce vasodilation and increase cardiac output in the human. The preferred method also comprises applying the heating patch assembly to a human chest.

### BRIEF DESCRIPTION OF THE DRAWINGS

The presently preferred embodiments of the invention are illustrated in the accompanying drawings, in which like reference numerals represent like parts throughout, and in which:

FIG. 1 is a front view of the preferred embodiment of the heating patch assemblies in accordance with the present invention shown on an exemplary human skeleton.

FIG. 1A is a front view of the preferred heating patch assemblies illustrated in FIG. 1 shown on an exemplary ribcage.

FIG. 2 is a back view of the preferred heating patch assembly illustrated in FIGS. 1-1A.

FIG. 3 is a sectional view of the preferred heating patch assembly illustrated in FIGS. 1-2.

### DESCRIPTION OF THE PREFERRED EMBODIMENTS OF THE INVENTION

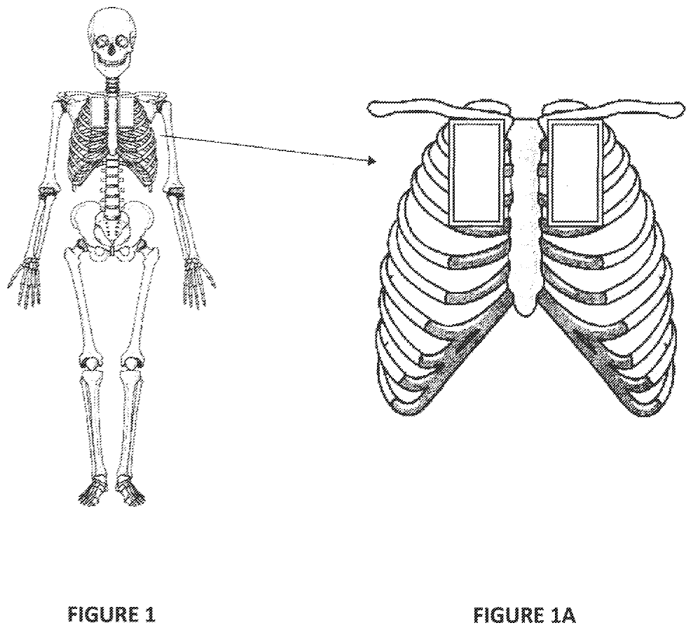

Referring now to the drawings, the preferred embodiment of the heating patch assembly in accordance with the present invention is illustrated by FIGS. 1 through 3.

Referring now to FIG. 1, a front view of the preferred embodiment of the heating patch assemblies in accordance with the present invention is shown on an exemplary human skeleton. As shown in FIG. 1, exemplary skeleton is designated generally by reference numeral 20, and the preferred heating patch assemblies are designated generally by reference numerals 30L and 30R. Preferred heating patch assembly 30L is disposed to the left of sternum 40, and preferred heating patch assembly 30R is disposed to the right of the sternum.

Referring now to FIG. 1A, a front view of preferred heating patch assemblies illustrated in FIG. 1 is shown on an exemplary ribcage. As shown in FIG. 1A, preferred heating patch assembly 30L is disposed to the left of sternum 40, and preferred heating patch assembly 30R is disposed to the right of the sternum. In addition, each of the preferred heating patch assemblies 30L and 30R are disposed just below left manubrium 50L and right manubrium 50R, respectively. Further, each of the preferred heating patch assemblies 30L and 30R extend downwardly away from the manubriums to an area above xiphoid process 60. In certain preferred embodiments, heating patch assembly 30R is disposed on a human chest adjacent to the human's right ventricle. In other preferred embodiments, heating patch assembly 30R is applied to the human chest adjacent to the first intercostal space and the second intercostal space to the right of the sternum between the manubrium and the angle of Louis in the sternum column.

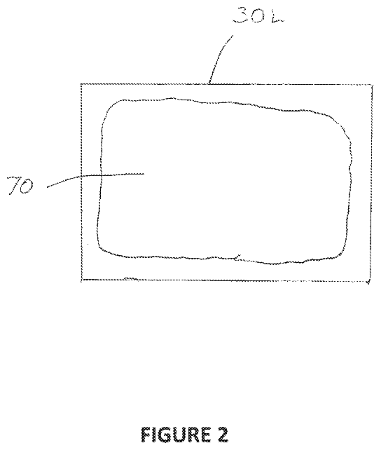

Referring now to FIG. 2, a back view of preferred heating patch assembly 30L is illustrated. As shown in FIG. 2, the back side of preferred heating patch assembly 30L comprises an adhesive such as gel 70 which is adapted to adhere the heating patch assembly to a human patient. While gel 70 is the preferred adhesive, it is contemplated within the scope of the invention that any suitable adhesive may be used such as double-sided tape.

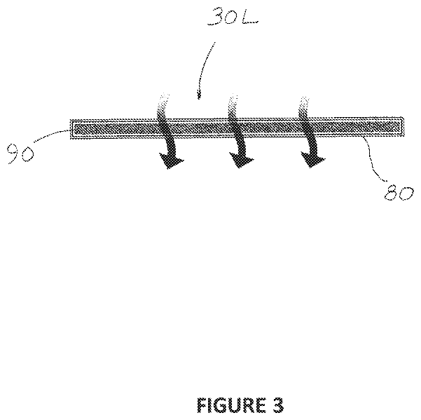

Referring now to FIG. 3, a sectional view of preferred heating patch assembly 30L is illustrated. As shown in FIG. 3, preferred heating patch assembly 30L comprises lining 80 and thermal conductor 90. Preferred thermal conductor 90 is disposed within preferred lining 80 In addition, preferred lining 80 is copper-infused so as to provide anti-microbial properties to the assembly. The preferred heating patch assembly thermal conductor is iron oxidation. Preferably, the thermal conductor produces a heating patch assembly temperature that is between approximately 38 degrees Celsius and approximately 40 degrees Celsius. Further, the preferred thermal conductor is adapted to produce heat for a period of approximately 2 hours to approximately 4 hours. The preferred heating patch assembly is adapted to induce vasodilation and increase cardiac output in the human.

The invention also comprises a method for increasing cardiac output. The preferred method comprises providing a heating patch assembly adapted for use on a human chest. The preferred heating patch assembly comprises a heating patch assembly lining and a heating patch assembly thermal conductor that is disposed within the heating patch assembly lining. The preferred assembly is adapted to induce vasodilation and increase cardiac output in the human. The preferred method further comprises applying the heating patch assembly to the human chest.

In other preferred embodiments, the method of the invention comprises a heating patch assembly that produces a temperature between approximately 38 degrees Celsius and approximately 40 degrees Celsius and is applied to the human chest for between approximately 2 hours and approximately 4 hours. In still other preferred embodiments, the method of the invention comprises a heating patch assembly that is applied to the human chest adjacent to the left ventricle and more particularly to the human chest adjacent to the first intercostal space and the second intercostal space to the left of the sternum between the manubrium and the angle of Louis in the sternum column.

Additionally, the preferred embodiments of the method of the invention comprise providing a second heating patch assembly that is applied to the human chest adjacent to the right ventricle.

In operation, several advantages of the preferred embodiments of the heating patch assembly are achieved. For example, the preferred embodiments of the heating patch assembly are non-invasive and create little to no risk to the patient. The preferred embodiments of the heating patch assembly are also unique in their shaping and construction. More particularly, rectangles on both sides of the chest that are a white tape substance with iron oxidation for material on the inside that lets off heat. They are 4 by 6 inches in order to properly cover the targeted arteries and veins. Conventional heating patch assemblies are not made for the area of the human body to which the preferred heating patch assembly is applied, and they are not adapted to provide the same targeted temperature range or heating range of time as the preferred heating patch assembly. Further, the preferred heating patch assembly includes a copper lining for anti-microbial purposes. Typically, after heart surgery there is a large wound down the middle of the chest where the sternum was cut so it is important to keep that area clean. Placement of the preferred patch assembly would start at the top of the manubrium and end above the xiphoid process, to the left and right of the sternum as shown in FIGS. 1 and 1A.

Additionally, it is common practice in cardiogenic shock, post-op heart recovery, and septic shock to monitor Cardiac Output and Cardiac Index. Cardiac Output (CO) is the volume of blood the heart pumps per minute, i.e., CO=Heart rate*Stroke volume. Cardiac Index (CI) is an assessment of cardiac output relative to Body Surface Area (BSA), i.e., CI=CO/BSA. Many things affect this outcome, but a key component is Systemic Vascular Resistance or afterload. It is in part the amount of force the left ventricle will have to overcome to open the aortic valve. Decreasing this number increases the Cardiac Output and the Cardiac Index. The aortic valve, the coronary arteries (coronary sinus), and innominate artery are all effected by heat. By providing a heat source that is on the skin and anatomically positioned over these areas, vasodilation may be induced. Once vasodilation is induced, more blood can flow to these areas and CO/CI increases. With the coronary arteries positioned posterior to the patch, they open allowing the heart to get an increase of blood flow. The preferred heating patch assembly allows the heart to meet cardiac demands more easily, thereby lowering the workload of the heart.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations, and the same are intended to be comprehended within the meaning and range of equivalents of the appended claims.

What is claimed is:

1. A heating patch assembly adapted for use on a human chest, said heating patch assembly comprising:
(a) a heating patch assembly lining;
(b) a heating patch assembly thermal conductor, said heating patch assembly thermal conductor being disposed within the heating patch assembly lining;
wherein the heating patch assembly is adapted to induce vasodilation and increase cardiac output in the human.

2. The heating patch assembly of claim 1 wherein the heating patch assembly lining is copper-infused.

3. The heating patch assembly of claim 1 wherein the heating patch assembly thermal conductor comprises iron oxidation.

4. The heating patch assembly of claim 1 further comprising a heating patch assembly adhesive.

5. The heating patch assembly of claim 1 wherein the heating patch assembly produces a temperature between approximately 38 degrees Celsius and approximately 40 degrees Celsius.

6. The heating patch assembly of claim 1 wherein the heating patch assembly produces heat for between approximately 2 hours and approximately 4 hours.

7. The heating patch assembly of claim 1 wherein the second heating patch assembly is applied to the human chest to the left of the sternum.

8. The heating patch assembly of claim 1 wherein the heating patch assembly is applied to the human chest adjacent to the left ventricle.

9. The heating patch assembly of claim 1 wherein the heating patch assembly is applied to the human chest adjacent to the first intercostal space and the second intercostal space to the left of the sternum between the manubrium and the angle of Louis in the sternum column.

10. The heating patch assembly of claim 1 further comprising a second heating patch assembly, said second heating patch assembly comprising:
(a) a second heating patch assembly lining;
(b) a second heating patch assembly thermal conductor, said second heating patch assembly thermal conductor being disposed within the second heating patch assembly lining;
wherein the second heating patch assembly is adapted to induce vasodilation and increase cardiac output in the human.

11. The heating patch assembly of claim 10 wherein the second heating patch assembly is applied to the human chest to the right of the sternum.

12. The heating patch assembly of claim 10 wherein the second heating patch assembly is applied to the human chest adjacent to the right ventricle.

13. The heating patch assembly of claim 10 wherein the second heating patch assembly is applied to the human chest adjacent to the first intercostal space and the second intercostal space to the right of the sternum between the manubrium and the angle of Louis in the sternum column.

14. A method for increasing cardiac output, said method comprising:
(a) providing a heating patch assembly adapted for use on a human chest, said heating patch assembly comprising:
(i) a heating patch assembly lining;
(ii) a heating patch assembly thermal conductor, said heating patch assembly thermal conductor being disposed within the heating patch assembly lining;
wherein the heating patch assembly is adapted to induce vasodilation and increase cardiac output in the human;
(b) applying the heating patch assembly to the human chest.

15. The method of claim 14 wherein the heating patch assembly produces a temperature between approximately 38 degrees Celsius and approximately 40 degrees Celsius.

16. The method of claim 14 wherein the heating patch assembly is applied to the human chest for between approximately 2 hours and approximately 4 hours.

17. The method of claim 14 wherein the heating patch assembly is applied to the human chest adjacent to the left ventricle.

18. The method of claim 14 wherein the heating patch assembly is applied to the human chest adjacent to the first intercostal space and the second intercostal space to the left of the sternum between the manubrium and the angle of Louis in the sternum column.

19. The method of claim 14 further comprising providing a second heating patch assembly.

20. The method of claim 14 wherein the second heating patch assembly is applied to the human chest adjacent to the right ventricle.
